(19) United States
(12) Patent Application Publication
(10) Pub. No.: US 2025/0140354 A1
(43) Pub. Date: May 1, 2025

(54) MODEL-AGNOSTIC EVALUATION OF A GENERATIVE MODEL IN MATERIALS DISCOVERY PROCESSES

(71) Applicant: International Business Machines Corporation, Armonk, NY (US)

(72) Inventors: Girmaw Abebe Tadesse, Nairobi (KE); Celia Cintas, Nairobi (KE); Jannis Born, Zürich (CH); Matteo Manica, Zürich (CH); Komminist Weldemariam, Ottawa (CA)

(21) Appl. No.: 18/385,725

(22) Filed: Oct. 31, 2023

(51) Int. Cl. G16C 20/70; G16C 20/80

(52) U.S. Cl. CPC G16C 20/70; G16C 20/80

(57) ABSTRACT

Domain-specific properties are extracted from a plurality of datasets related to input molecules and generated molecules. A similarity of a given one of the generated molecules with a candidate molecule is evaluated and the evaluated similarity is aggregated across multiple constraints to generate a single score. An ability of a given generative model to mimic the input molecules is quantified based on an aggregation of scores for multiple properties. The generative models are ranked by their ability to mimic the input molecules and evaluation properties based on the aggregated scores. One or more rates in the generated molecules are quantified based on the aggregated single score. One of the generative models is selected based on the ranking and a new molecule is generated using the selected generative model.

### STATEMENT REGARDING PRIOR DISCLOSURES BY THE INVENTOR OR A JOINT INVENTOR

The following disclosure(s) are submitted under 35 U.S.C. 102(b)(1)(A):

Girmaw Abebe Tadesse, Jannis Born, Celia Cintas, William Ogallo, Dmitry Zubarev, Matteo Manica, and Komminist Weldemariam, Domain-agnostic and Multi-level Evaluation of Generative Models, arXiv preprint arXiv:2301.08750v1, 2023 Jan. 20.

### BACKGROUND

The present invention relates generally to the electrical, electronic and computer arts and, more particularly, to machine learning.

Generative models are used to accelerate discovery across various domains including drug discovery, climate change, and the like. Though the generation capability has been improved using larger training data and sophisticated training methodologies, the evaluation of generated samples and comparative evaluation of different generative models remains a challenge.

Cheminformatics is an area of science built upon the fields of chemistry, computer science, and information science, and is often applied to biology and related molecular fields. Cheminformatics may be used in the process of drug discovery, such as structure-based drug design, by pharmaceutical companies and in academia. Cheminformatics may be applied to the storage, indexing, and search of information for chemical compounds where the chemical data can be related to real or virtual molecules. Virtual libraries of compounds are readily available for use in the process of drug discovery.

High-throughput screening is an experimentation method used in drug discovery, biology, and chemistry that enables millions of chemical tests, pharmacological tests, and the like to be quickly conducted. The results of the tests can be used in drug design. Virtual screening, on the other hand, is able to identify candidates likely to exhibit target properties, such as a biological effect on a given target.

### BRIEF SUMMARY

Principles of the invention provide systems and techniques for model-agnostic evaluation of a generative model in materials discovery processes. In one aspect, an exemplary method includes the operations of extracting, using at least one hardware processor, domain-specific properties from a plurality of datasets related to input molecules and generated molecules; evaluating a similarity of a given one of the generated molecules with a candidate molecule; aggregating, using the at least one hardware processor, the evaluated similarity across multiple constraints to generate a single score; quantifying, using the at least one hardware processor, an ability of a given generative model to mimic the input molecules based on an aggregation of scores for multiple properties; ranking, using the at least one hardware processor, the generative models by their ability to mimic the input molecules and evaluation properties based on the aggregated scores; quantifying, using the at least one hardware processor, one or more rates in the generated molecules based on the aggregated single score; selecting, using the at least one hardware processor, one of the generative models based on the ranking; and generating, using the at least one hardware processor, a new molecule using the selected generative model.

In one aspect, a computer program product comprises one or more tangible computer-readable storage media and program instructions stored on at least one of the one or more tangible computer-readable storage media, the program instructions executable by a processor, the program instructions comprising extracting domain-specific properties from a plurality of datasets related to input molecules and generated molecules; evaluating a similarity of a given one of the generated molecules with a candidate molecule; aggregating the evaluated similarity across multiple constraints to generate a single score; quantifying an ability of a given generative model to mimic the input molecules based on an aggregation of scores for multiple properties; ranking the generative models by their ability to mimic the input molecules and evaluation properties based on the aggregated scores; quantifying one or more rates in the generated molecules based on the aggregated single score; selecting one of the generative models based on the ranking; and generating a new molecule using the selected generative model.

In one aspect, an apparatus comprises a memory and at least one processor, coupled to the memory, and operative to perform operations comprising extracting domain-specific properties from a plurality of datasets related to input molecules and generated molecules; evaluating a similarity of a given one of the generated molecules with a candidate molecule; aggregating the evaluated similarity across multiple constraints to generate a single score; quantifying an ability of a given generative model to mimic the input molecules based on an aggregation of scores for multiple properties; ranking the generative models by their ability to mimic the input molecules and evaluation properties based on the aggregated scores; quantifying one or more rates in the generated molecules based on the aggregated single score; selecting one of the generative models based on the ranking; and generating a new molecule using the selected generative model.

As used herein, “facilitating” an action includes performing the action, making the action easier, helping to carry the action out, or causing the action to be performed. Thus, by way of example and not limitation, instructions executing on a processor might facilitate an action carried out by instructions executing on a remote processor and/or steps carried out by chemical process equipment, by sending appropriate data or commands to cause or aid the action to be performed. Where an actor facilitates an action by other than performing the action, the action is nevertheless performed by some entity or combination of entities.

Techniques as disclosed herein can provide substantial beneficial technical effects. Some embodiments may not have these potential advantages and these potential advantages are not necessarily required of all embodiments. By way of example only and without limitation, one or more embodiments may provide one or more of:
evaluation of the similarity or divergence of generated molecules relative to training molecules and/or between molecules generated from different models (per a property or structural feature);
a single score that captures the similarity or divergence of molecules across multiple constraints (such as chemical properties and structural details);
quantification of how well a generative model for molecules mimics training data based on an aggregation of scores for multiple properties using normalized odds ratio, a distribution-similarity metric or both;
a ranking of generative models by their ability to mimic a molecule training dataset and required novelty/divergence evaluation properties based on the aggregated scores of all properties;
quantification of validity, uniqueness, and novelty rates in generated molecules based on a principally aggregated similarity or divergence score;
methods to detect, characterize, and quantify novelty as a principled aggregation of molecule properties and structural requirements;
methods to improve a generation process for molecules that aims to characterize and quantify how capable a generative model is for generating a certain type of candidate molecule for a given task and, with this information, informing a user which model to use for a given type of property needed in the generated molecule as well as informing which models to use during inference time to control the generation process;
methods to improve the interpretability of different generative models as well as comparison between generative processes and the generated new candidates molecules and enable a domain-expert to improve the molecule generation process (output comparison allows comparison of different generative processes and their generated candidate molecules for a given task);
methods to detect and characterize systemic error property predictors (bias by under- or over-predicting for a particular set of molecules) and mitigate its propagation to the generation process;
a principled approach that allows the assessment of how well any generative model reflects certain properties of the training data;
naturally scales to multiple properties of molecules; and
metrics that can easily be extended to capture “objectives.”

These and other features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1 is a block diagram of a data pipeline for an example model-agonistic evaluation framework, in accordance with example embodiments;

FIG. 2 is an example workflow for evaluating the generated molecules compared to training molecules and ranking the generative models accordingly, in accordance with example embodiments;

FIG. 3A illustrates details of a multi-level evaluation component that includes Hierarchical Independence Evaluation (HIE) and Generation Frequency Analysis (GAF), in accordance with an example embodiment;

FIG. 3B is an example customized multi-dimensional subset scanning method for evaluating one or more generative models used in a materials discovery process, in accordance with example embodiments;

FIG. 4 is a flowchart for performing the model-agnostic evaluation of a generative model in materials discovery process, in accordance with example embodiments;

FIG. 5 is a flowchart for an example method for a modeling performance improvement process, in accordance with example embodiments;

FIG. 6A is a bar chart illustrating the scanning results on the generated molecules, in accordance with example embodiments;

FIG. 6B is a table illustrating the results of a comparative evaluation of training a conventional graph convolutional policy network (GCPN) and a conventional flow-based autoregressive model for molecules per their quantum electrodynamic (QED) values, in accordance with example embodiments;

FIG. 7 is a graph providing a visualization of the quantum electrodynamics (QED) distributions of training with real molecules and molecules generated from graph convolutional policy network (GCPN) and Graph models, in accordance with example embodiments;

FIG. 8A is a table of Hierarchical Independence Evaluation (HIE) scores for GCPN models and conventional flow-based autoregressive models for molecular graph generation trained on a first conventional dataset, in accordance with example embodiments;

FIG. 8B is a table of Hierarchical Independence Evaluation (HIE) scores for GCPN models and conventional flow-based autoregressive models for molecular graph generation trained on a second conventional dataset, in accordance with example embodiments; and

FIG. 9 depicts a computing environment according to an embodiment of the present invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of inventions described herein will be in the context of illustrative embodiments. Moreover, it will become apparent to those skilled in the art given the teachings herein that numerous modifications can be made to the embodiments shown that are within the scope of the claims. That is, no limitations with respect to the embodiments shown and described herein are intended or should be inferred.

Generally, techniques are provided for evaluating generative models in materials discovery processes without accessing the latent details. In one example embodiment, domain-specific chemical and biological properties are extracted from a plurality of datasets related to molecules (such as conventional datasets of existing physical molecules) and generated examples of molecules. (It is noted that the generated molecules are not necessarily physically generated.) The similarity or divergence of the generated molecules from training molecules and/or between molecules generated from different models are evaluated per a property or structural feature. The similarity or divergence across multiple constraints (such as chemical properties and structural details) are aggregated into a single score. How well a generative model mimics the training data is quantified based on an aggregation of scores for multiple properties using normalized odds ratio, a distribution-similarity metric, or both. Multiple generative models are ranked by their ability to mimic the training dataset and required novelty/divergence evaluation properties based on the aggregated scores of all properties. The validity, uniqueness, and novelty rates in the generated molecules are quantified based on the principally aggregated similarity or divergence score. The divergence score for a subgroup of samples candidates S with a reference dataset D is computed as:

$$Γ ( 𝒮 , 𝒟 ) = {max}_{q} log ( q ) {\sum }_{i ∈ 𝒮} {y}_{i} - {N}_{s} * log ( 1 - {e}_{g} + q {e}_{g} )$$

where y is an outcome label, N is the number of samples, and e is an expectation.

The Generation Frequency Analysis aims to identify a group of samples with extreme deviations in their generation rate compared to

$${e}_{g} = \frac{{N}_{g}}{{N}_{g} + {N}_{t}} ,$$

(N_{g} corresponds to the number of generated samples and Nₜ corresponds to the number of real/training samples). The deviations between expectation and observation are evaluated by maximizing the Bernoulli likelihood ratio scoring statistic (Γ), defined above.

Conventional generative models are used to accelerate discovery across various domains including drug discovery, climate change, and the like. Though the generation capability has been improved using larger training datasets and sophisticated training methodologies, evaluation of generated samples and comparative evaluation of different generative models remains a challenge:
driven by inaccurate evaluation metrics that oversimplify real discovery problems (such as drug-likeness, synthesizability, and the Lipinski rule);
split into distributional metrics (such as uniqueness and diversity) and property-based metrics; that is, scoring individual molecules (It is noted that conventional techniques lack the methodology to combine both: a widely accepted and common-sense approach to measure the distributional similarity of generated molecules in comparison to input molecules 216 (also referred to as input dataset 216 herein) for particular properties. For example, if one is not interested in any properties, the samples can be directly compared using maximum-mean discrepancy (MMD) on feature descriptor);
less practical techniques or techniques that use a synthetic metric (such as logarithmic partition coefficient (Log P) and drug-likeness (QED));
focused on a single-constraint-objective (such as solubility) without a principled approach to incorporate multiple chemical target properties;
dependent on flawed predictive models for goal oriented or constrained generation (such as a trained toxicity predictor is used to estimate the toxicity level of the generated molecules to evaluate the quality of generation. However, if there is a systematic error, such as over or under prediction for a specific set of molecules, in the prediction model, it could be propagated to the generative process resulting in less useful/practical generation of molecules); and
model dependent (such as a conventional metric for generative models for drug design, which depends on a latent representation in a neural network model that a particular input causes (such metrics suffer from limited interpretability by domain experts in addition to being modality- and/or model-specific)).

Furthermore, one or more embodiments address gaps in the prior art including:
model specific and, hence, specific to a particular chemical/physical property;
lack of interpretability (domain-expert-in-the-loop) especially for latent-based evaluation metrics such as dimensionless metrics like MMD;
distribution-learning metrics could be less informative in the practical use of the generated molecules;
goal-directed metrics could easily exploit biases in the scoring functions;
current metrics can be easily fooled by simplistic generative models like the “add-carbon-algorithm” (which generates molecules by randomly adding carbons to training data samples); and
no systematic metric that is naturally multi-objective: “real molecular design problems are generally multi-objective in nature.”

Example embodiments provide a multi-level and domain-agnostic evaluation of generative models, starting with sub-feature or feature-based low-level evaluation to global features based high level-evaluation. In example embodiments, the generation capability of each black box model is evaluated without any information about its generative process, in a scalable manner (and possibly multi-objective manner; i.e., evaluation of the model may be based on more than one evaluative objective; for example, the objective may include multiple properties) and with interpretable description of the evaluation. The input includes: 1) one or more sets of data samples that were generated by the black-box generative models; 2) one or more sets of training data samples; and 3) a user-defined list of properties that can be computed from the generated and training data samples.

One or more exemplary embodiments advantageously meet the need of model-agnostic evaluation of generated samples and models with:
1) ability to apply across different input modalities and generative models;
2) ability to generalize across different outcomes of interests including properties and structural details;
3) methods to detect, characterize, and quantify novelty as a principled aggregation of these properties and structural requirements;
4) methods to improve the interpretability of the comparison output and enable a domain-expert to improve the generation process;
5) methods to detect and characterize systemic error property predictors (bias by under- or over-predicting for a particular set of molecules) and mitigate its propagation to the generation process;
6) ability to better reflect the complexity of real discovery problems; and
7) a principled approach that allows researchers to assess how well any generative model reflects certain properties of the training data.

In example embodiments, generative models in a materials discovery process are evaluated without accessing the latent details while providing an interpretable description of the evaluation. The system considers different input modalities, different generative models and different outcomes of interests, including properties and structural details. More specifically, the disclosed system uses one or more sets of data samples that were generated by black-box generative models (such as Flow-based Autoregressive Models, Graph Convolutional Networks, Variational Autoencoders, Generative Adversarial Networks, and the like), one or multiple sets of training data samples and a user-defined list of properties that can be computed from the generated and training data samples. Pretrained machine learning models (such as a conventional cheminformatics toolkit, as will be familiar to a person having ordinary skill in the art) are used to automatically extract domain-specific chemical and biological properties from a given plurality of datasets related to molecules and generated examples of molecules.

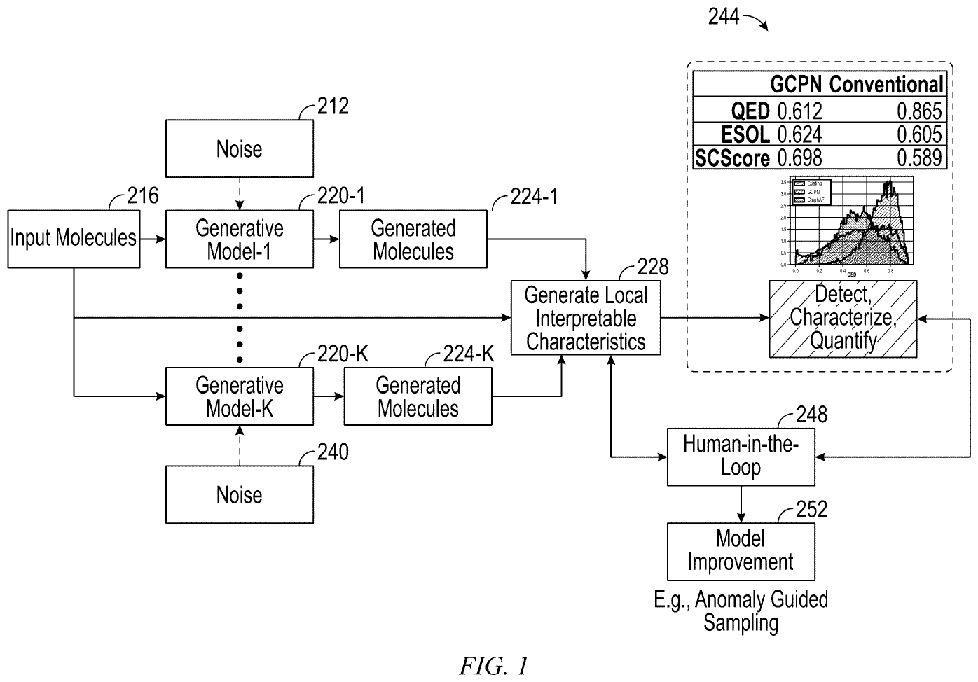

FIG. 1 is a block diagram of a data pipeline for an example model-agonistic evaluation framework, in accordance with example embodiments. In one example embodiment, generative model 220-1, . . . , 220-K are neural networks and input molecules 216 and noise variables 212, 240 are input to each generative model 220-1, . . . , 220-K, which produce sets of generated molecules 220-1, . . . , 220-K. The generative models 220-1, . . . , 220-K use the noise variables (whose values are sampled from a given distribution) to create the generated molecules 220-1, . . . , 220-K. In one example embodiment, this is part of an initialization step when creating the generated molecules 220-1, . . . , 220-K. Thus, both noise variables 212 and 240 may be used as initialization steps where these two variables are sampled from two different distributions.

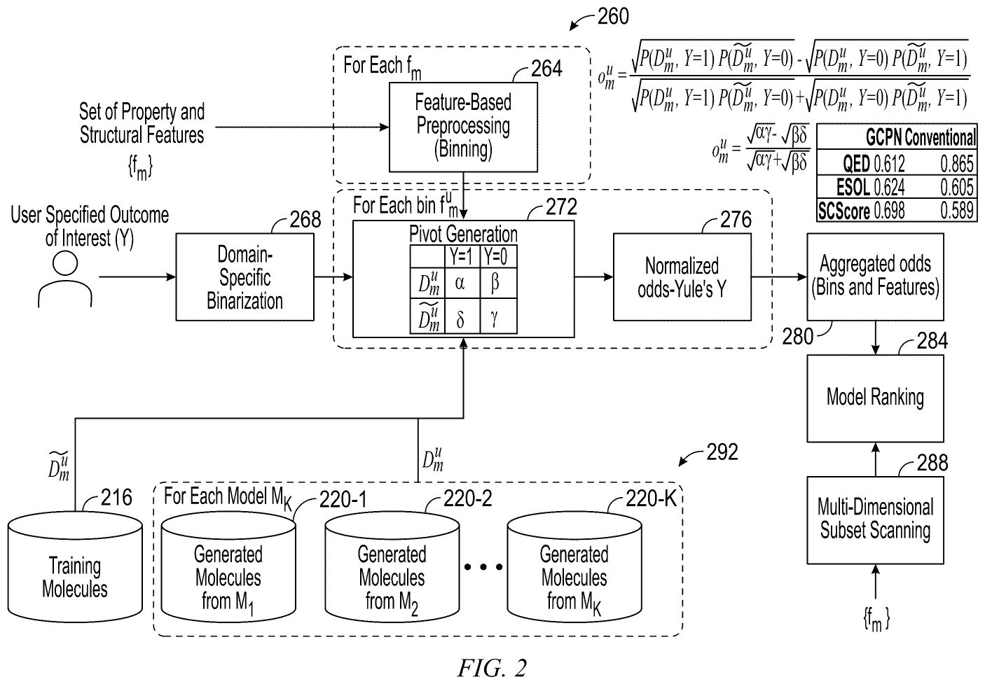

The input molecules 216 and the generated molecules 220-1, . . . , 220-K are used to generate local interpretable characteristics (operation 228) and the generated local interpretable characteristics are detected, characterized and quantified by process 244 (see FIG. 2). (As noted above, a conventional cheminformatics toolkit, as will be familiar to a person having ordinary skill in the art, may be used to generate the local interpretable characteristics. An individual may also specify functionality for generating the local interpretable characteristics for other types of features.) A human-in-the-loop 248 may participate in improving the model via the model improvement module 252 based on the generated local interpretable characteristics and the results of process 244.

FIG. 2 is an example workflow for evaluating the generated molecules 224-1, . . . , 224-K compared to input molecules 216 and ranking 284 the generative models 220-1, 220-2, . . . , 220-K accordingly, in accordance with example embodiments. In one example embodiment, the workflow of FIG. 2 implements the process 244 and runs on a general purpose computer, a desktop computer, a server, or the like. In one example embodiment, the model-agnostic system of FIG. 2 evaluates the generation capability of each black-box model 220-1, 220-2, . . . , 220-K without any information about its generative process.

Feature-based preprocessor 264 preprocesses each feature where, for example, the type of feature values could be binary, continuous, categorical, and the like. (In one example embodiment, further pre-processing is applied in the follow-up steps.) For example, discretization of continuous features is required for sub-feature-level performance evaluation. Example embodiments provide different discretization types, such as equal width, equal frequency, k-means based, and the like. Domain-specific binarization 268 performs a preprocessing step associated with binarizing the outcome proposed by a user for a given task. The binarization may be adapted based the type of outcome. For example, a user may input certain characteristics and/or features of the desired molecule. It is noted that the process of binarization will be familiar to the skilled artisan.

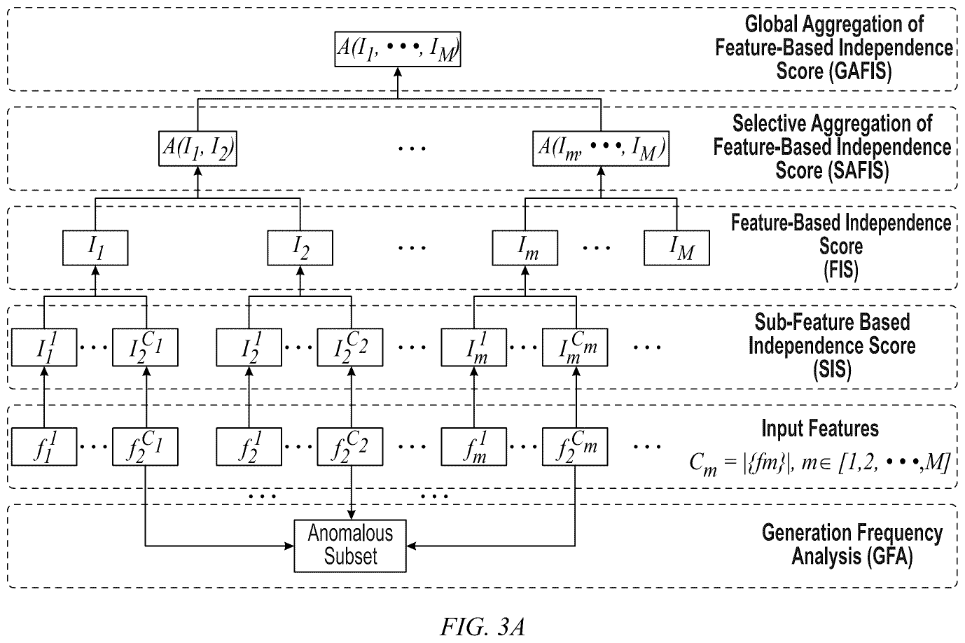

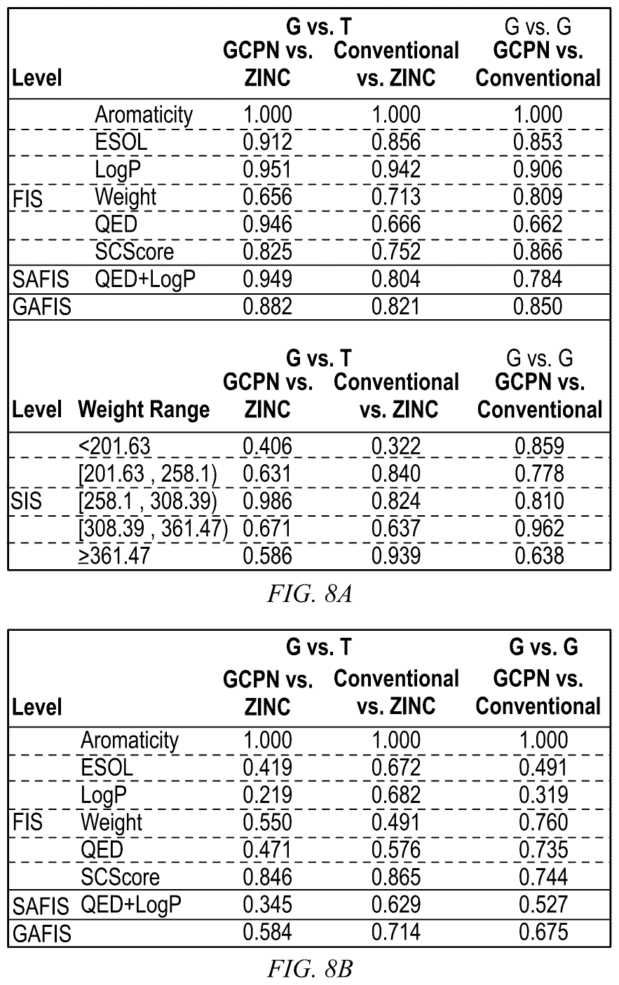

FIG. 3A illustrates details of a multi-level evaluation component that includes Hierarchical Independence Evaluation (HIE), such as SIS, FIS, SAFIS, GAFIS, and the like, and Generation Frequency Analysis (GAF), in accordance with an example embodiment. A( . . . ) represents an aggregation operation, and anomalous subset refers to the logical combinations of features that characterize samples generated with extreme frequencies. As illustrated in FIG. 3A, HIE follows a bottom-up approach (from a sub-feature to global aggregation levels) and evaluates the performance of the generative models 220-1, 220-2, . . . , 220-K at different levels of feature interactions. The lowest level of evaluation in HIE is the sub-feature level independence score (SIS), which aims to quantify the performance of the generative models 220-1, 220-2, . . . , 220-K across different ranges or unique values per feature, e.g., based on a molecular weight range of [200, 300) Daltons. The second layer in HIE represents a feature-level independence score (FIS), which quantifies the generation performance for each feature. To this end, FIS can be computed via aggregation of SIS values, thereby providing a weighting strategy for SIS scores per feature. FIS values can also be computed directly, without aggregating SIS values, using a different choice of objective measure directly applied on the whole feature. Examples of these HIEs can be found in FIGS. 8A and 8B.

Returning to FIGS. 1-2, the system evaluates the similarity or divergence of the generated molecules 224-1, . . . , 224-K from the input molecules 216 and/or between molecules 224-1, . . . , 224-K generated based on different models 220-1, 220-2, . . . , 220-K per a property or structural feature at different levels, as described above and shown in FIG. 3A. That is, once the domain-specific features are extracted, evaluation is performed for each feature via a normalized odds ratio of each feature value (such as aromatic structure) between the two sets of molecules 216, 220-1, 220-2, . . . , 220-K, such as input molecules 216 vs. generated molecules 220-1, 220-2, . . . , 220-K by model (operation 276). Yule's coefficient is an example of a normalized odds ratio employed due to its capability to satisfy a number of required criteria for objective measures, such as symmetry under variable permutation, scaling and inversion invariances. To this end, a 2×2 pivot table is generated (operation 272) as:

| | Feature exists | Feature does not exist |
|---|---|---|
| Set of molecules generated | a | b |
| from model K | | |
| Set of training molecules | c | d |

Then the Yule's Y coefficient is computed as:

$$\begin{matrix}{o}_{m}^{u} = \frac{\sqrt{P ( {D}_{m}^{u} , Y = 1 ) P ( \tilde{{D}_{m}^{u}} , Y = 0 )} - \sqrt{P ( {D}_{m}^{u} , Y = 0 ) P ( \tilde{{D}_{m}^{u}} , Y = 1 )}}{\sqrt{P ( {D}_{m}^{u} , Y = 1 ) P ( \tilde{{D}_{m}^{u}} , Y = 0 )} - \sqrt{P ( {D}_{m}^{u} , Y = 0 ) P ( \tilde{{D}_{m}^{u}} , Y = 1 )}} \\ {o}_{m}^{u} = \frac{\sqrt{α γ} - \sqrt{β δ}}{\sqrt{α γ} + \sqrt{β δ}}\end{matrix}$$

Given a feature and its different unique values, their Yule's coefficients are aggregated weighted on a user-specified and/or data-driven importance score of each feature value after quantization into a specified number of bins (operations 272-280; the Yule's coefficients are calculated from the pivot table generated in operation 272).

The system aggregates the similarity or divergence across multiple constraints (such as chemical properties and structural details) into a single score (see description of FIG. 3A) and quantifies how well a generative model 220-1, 220-2, . . . , 220-K mimics the input data 216 based on aggregated scores. This aggregation of scores aims to perform the quantification in a standard-way, taking into consideration multiple objectives that need to be satisfied. Principled aggregation is employed using a data-driven and/or domain-specific evaluation of different chemical properties and structures. A customized multi-dimensional subset scanning method 288 is used for evaluating the one or more generative models 220-1, 220-2, . . . , 220-K used in materials discovery processes (see FIG. 3B). The scanning approach is a novel process to identify if there is a subset of molecules (training or generated) that exhibit similar or divergent characteristics of the outcome of interest. For example, determining which kind of molecules a generative model 220-1, 220-2, . . . , 220-K often generates. These subsets of molecules are also referred to as anomalous subsets. The similarity or divergence of the generated molecules 220-1, 220-2, . . . , 220-K from the input molecules 216 and/or between molecules 220-1, 220-2, . . . , 220-K generated from different models 220-1, 220-2, . . . , 220-K is evaluated.

FIG. 3B is an example customized multi-dimensional subset scanning (MDSS) method 288 for evaluating one or more generative models 220-1, 220-2, . . . , 220-K used in a materials discovery process, rendered in pseudo-code, in accordance with example embodiments. A goal of subgroup discovery is to identify descriptions of data subsets that show an interesting distribution with respect to a pre-specified target concept. The Generation Frequency Analysis algorithm in FIG. 3B may be used to implement the multi-dimensional subset scanning method 288. In one example embodiment, automated stratification of samples using multi-dimensional subset scanning (MDSS) is used to identify a subset of samples generated with divergent frequencies (other methods, such as a conventional automatic data slicing framework, a conventional subgroup discovery package, and the like are also contemplated for implementing the multi-dimensional subset scanning method 288). As illustrated in FIG. 3B, lines 1-3 initialize the returned subset to empty, a low score for the anom (anomalous) score and a converge flag set to False. In the while loop (from lines 4 to 13), a random subset of records is selected to obtain an abnormal score and determine which subset of features is responsible for that score (lines 6-7). If the score of the newly found subset is greater than the previous score, both the scores and the new return set are updated (lines 8-11); if greater scores cannot be found, the while loop is exited, and the highest score and subset are returned (line 14).

In one example embodiment, the multiple generative models 220-1, 220-2, . . . , 220-K are ranked by their ability to mimic the input dataset 216 (input molecules 216) and required novelty/divergence evaluation properties based on the aggregated scores of all properties. The ranking 284 is performed as per the required characteristics of model performance, whether it is aggregated properties (such as toxicity), structural details (such as aromatic string and number of rings), and the like. (In one example embodiment, the ranking 284 is based on the aggregated score (aggregated odds).) How well a generative model 220-1, 220-2, . . . , 220-K mimics the input dataset 216, based on an aggregation of scores for multiple properties, is then quantified. Using the aggregated scores of all properties, multiple generative models 220-1, 220-2, . . . , 220-K are then ranked by their ability to mimic the properties of the input dataset 216. The aggregated similarity or divergence score is then used to quantify the validity, uniqueness, and novelty rates in the generated molecules 224-1, . . . , 224-K. This way, the one or more generative models 220-1, 220-2, . . . , 220-K with the higher scores will be ranked at the top and vice versa.

Using the customized multi-dimensional subset scanning method 288 of FIG. 3B, the disclosed system also analyzes predictive models designed to predict certain properties of molecules, such as solubility and the like, for potential systemic deviations, such as over-predicting for a particular set of molecules. The scanning method 288 enables the automatic identification bias in prediction of models efficiently via an additive linear time subset scanning property. The findings are used to mitigate bias in these models and the aggregation of scores down weight the impact of these models on the final scoring.

Once the generative models 220-1, 220-2, . . . , 220-K are characterized and ranked based on aggregated or specific performance, the top performing generative model 220-1, 220-2, . . . , 220-K may be selected to generate the molecules 224-1, . . . , 224-K with the required constraint, such as “Model B” is best in generating molecules 224-1, . . . , 224-K with similar structural information as the input dataset 216 whereas “Model C” is the best in generating molecules 224-1, . . . , 224-K with reduced toxicity. Moreover, the findings may be used as a feedback loop, together with domain-expert insights, to further improve the generation performance of a particular generative model 220-1, 220-2, . . . , 220-K.

In one example embodiment, quantifying the validity, uniqueness, and novelty rates in the generated molecules 224-1, . . . , 224-K is based on the principally aggregated similarity or divergence score. More specifically, the validity is computed with the conventional cheminformatics toolkit, and novelty and uniqueness of the generated molecules 224-1, . . . , 224-K are defined with respect to the input dataset 216 and the generated dataset 224-1, . . . , 224-K (the generated molecules 224-1, . . . , 224-K), respectively. Note also that the validity, uniqueness, and novelty metrics can be computed for each molecule 224-1, . . . , 224-K; therefore, the validity, uniqueness, and novelty metrics can be used just like any other feature. For the input molecules 216, validity and uniqueness will always be 1 (unless the input dataset 216 contains errors) and novelty will always be 0 (by definition).

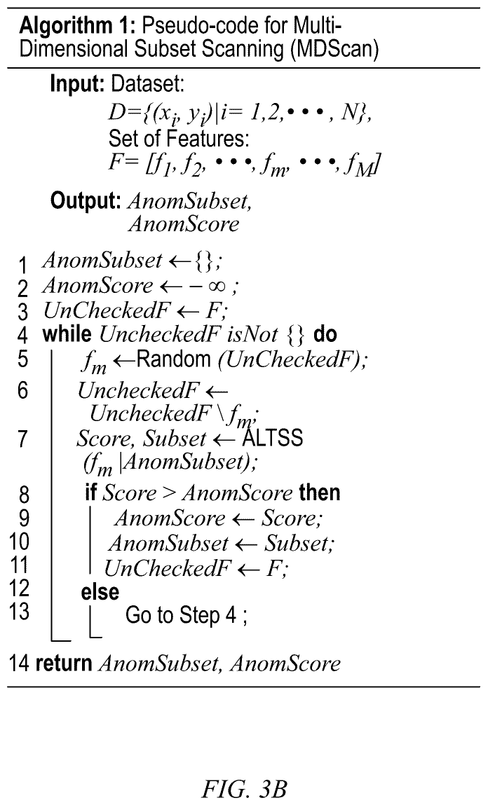

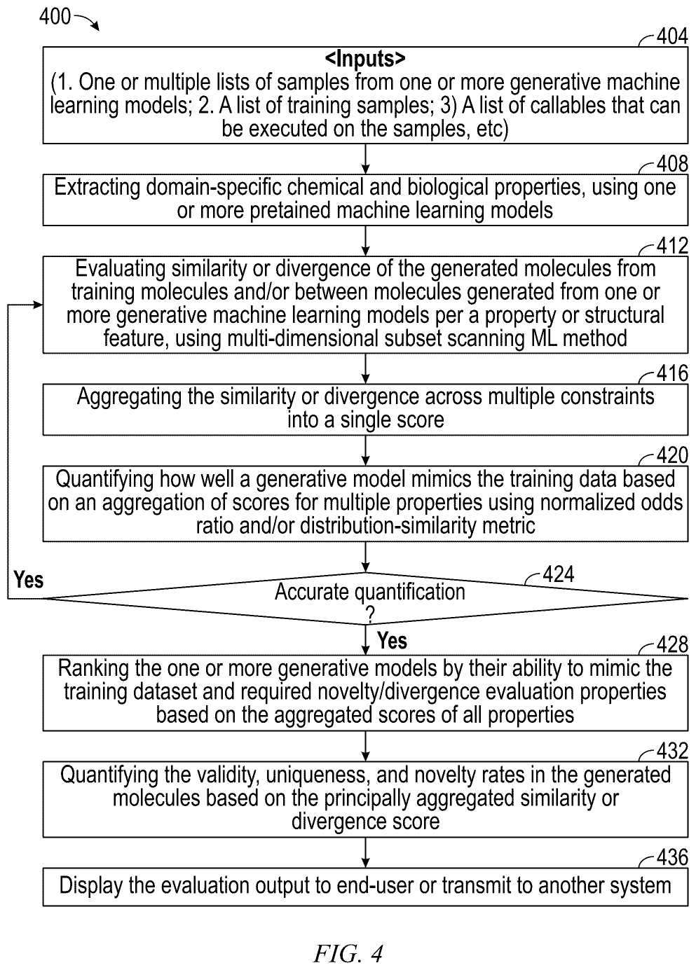

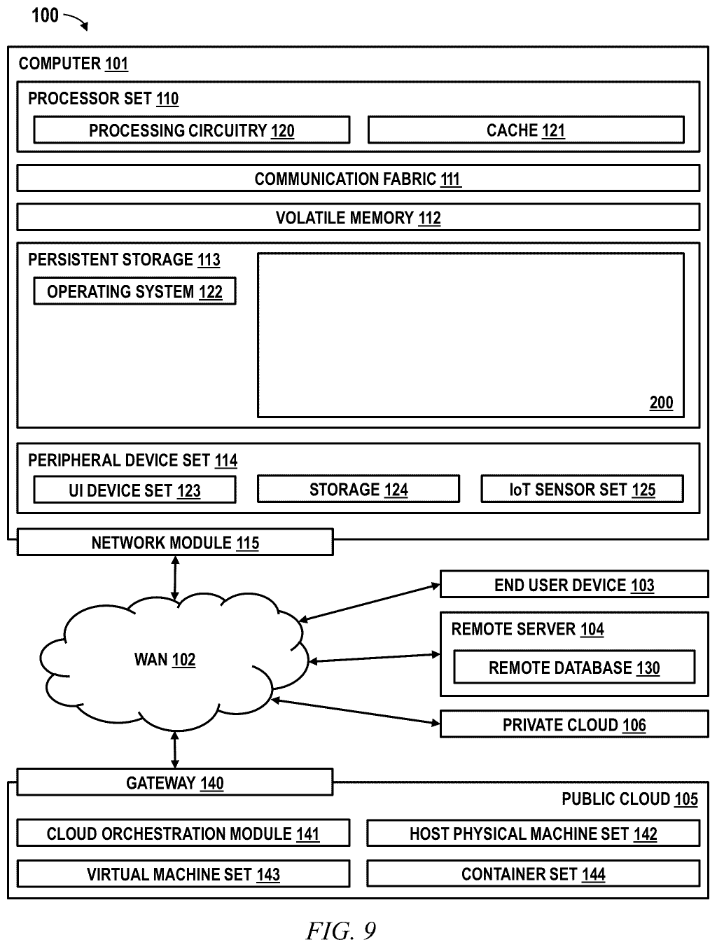

In one example embodiment, the model-agonistic evaluation framework includes the following modules (implemented by the flowcharts of FIGS. 4-5 running, for example, on the computing environment of FIG. 9):

Data/Property Extraction Module 228 (see operation 408 of FIG. 4): this module receives lists of samples from multiple generative models 220-1, 220-2, . . . , 220-K, a list of input samples 216 and a list of score functions that characterize a set of properties from the samples for a given domain. For example, given molecule information, e.g., Simplified Molecular Input Line Entry System (SMILES), extraction of different features and properties (both chemical and structural) can be performed with chemoinformatics libraries (such as the conventional cheminformatics toolkit). Classic properties measurements are molecular weight, scaffolding, ring and aromatic structures. Further properties can include drug-likeness (QED), synthetic complexity metric (SCScore), or other application-specific properties that can be computed in-silico. Additionally, the user can provide the function to be used to compute a custom/domain specific property. The feature extraction model is general enough to work on any modalities (e.g., can work on SMILES strings, images, and the like) if the provided score functions work on images, including binary, categorial or continuous features.

Customize multi-dimensional subset scanning module 288: this module takes all the properties extracted across different samples and generative models 220-1, 220-2, . . . , 220-K and determines whether there is a subset of molecules (input molecules 216 or generated molecules 224-1, . . . , 224-K) that exhibit divergent characteristics of the outcome of interest, such as which kind of molecules 224-1, . . . , 224-K a generative model 220-1, 220-2, . . . , 220-K often generates. These subsets of molecules are also referred to as anomalous subsets. The pseudocode for this process is shown in FIG. 3B.

Interactive module (human-in-the-loop) 248 (implementing operation 508, as described below in conjunction with FIG. 5): provides a way to visualize a complex set of properties, divergence scores and generation rules so that the domain expert can assess the results found by the disclosed method. The detection of a divergent subset of molecules using scanning over the previously extracted properties and structural features helps to bridge domain-expert insight for hypothesis generation and validation of the MDSS-based findings.

Model improvement module 252 (see operations 512-516 of FIG. 5): the validated findings and domain-expert-input rules are used for the generation process. During training, these terms can be used as penalization weights for a given subset of samples in the objective function. Further, at inference time, this set of rules can be used as a sampling criteria. Both of these feedback loop interventions can help generative models 220-1, 220-2, . . . , 220-K to achieve better performance in sample generation.

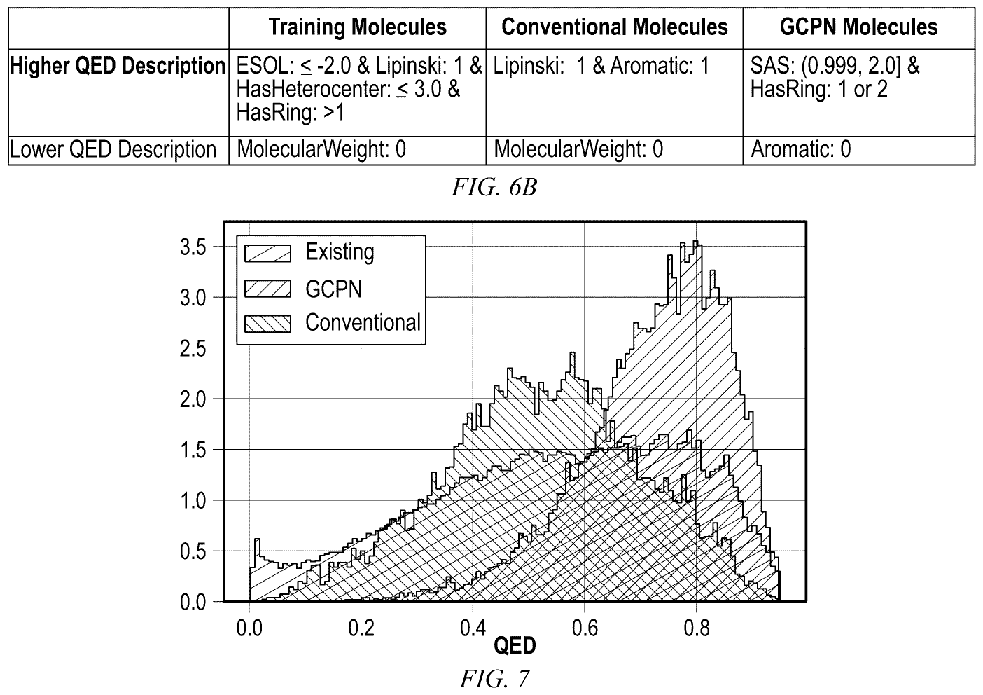

FIG. 4 is a flowchart 400 for performing the model-agnostic evaluation of a generative model 220-1, 220-2, . . . , 220-K in materials discovery processes, in accordance with example embodiments. Inputs 404 include: 1) one or more lists of samples from one or more generative models 220-1, 220-2, . . . , 220-K; 2) a list of training samples; and 3) a list of callables that can be executed on the samples and the like. In one example embodiment, domain-specific chemical and biological properties are extracted using one or more pretrained machine learning models (operation 408). Similarity or divergence of the generated molecules 224-1, . . . , 224-K from input molecules 216 and/or between molecules 224-1, . . . , 224-K generated from one or more generative models 220-1, 220-2, . . . , 220-K are evaluated using a multi-dimensional subset scanning machine learning (ML) method per a property or structural feature (operation 412). The similarity or divergence across multiple constraints are aggregated into a single score (operation 416). (The constraints are illustrated in FIG. 3A as different levels, as described above.) How well a generative model 220-1, 220-2, . . . , 220-K mimics the input dataset 216 based on an aggregation of scores for multiple properties using a normalized odds ratio and/or distribution-similarity metric is quantified (operation 420; see, for example, FIG. 7).

A check is then performed to determine the status of the quantification (operation 424). For example, as depicted in FIG. 7, if a generative model 220-1, 220-2, . . . , 220-K generates molecules that are sufficiently similar to existing molecules (such as a confidence level of 95% or 98%), the quantification is considered accurate. If the quantification is not accurate (no branch of decision block 424), the method proceeds with operation 412; otherwise (yes branch of decision block 424), the one or more generative models 220-1, 220-2, . . . , 220-K are ranked by their ability to mimic the input dataset 216 and required novelty/divergence evaluation properties based on the aggregated scores of all properties (operation 428). The validity, uniqueness, and novelty rates in the generated molecules 224-1, . . . , 224-K are quantified based on the principally aggregated similarity or divergence score (operation 432). (Given the teachings herein, the skilled artisan can employ conventional techniques for quantifying such rates.) The evaluation output to end-user is displayed or transmitted to another system (operation 436).

FIG. 5 is a flowchart 500 for an example method for a modeling performance improvement process, in accordance with example embodiments. In one example embodiment, a detected divergent subset of molecules is generated using scanning over the previously extracted properties and structural features (operation 504; see FIG. 3B and the model improvement module 252). The generated divergent subset of molecules is displayed on a user device and domain-expert input/rules for model improvement are received (operation 508). Model improvement features (parameters, constraints, rules and the like) are determined (operation 512) and a model retraining and/or inferencing process is triggered (operation 516).

### Validation Use Case

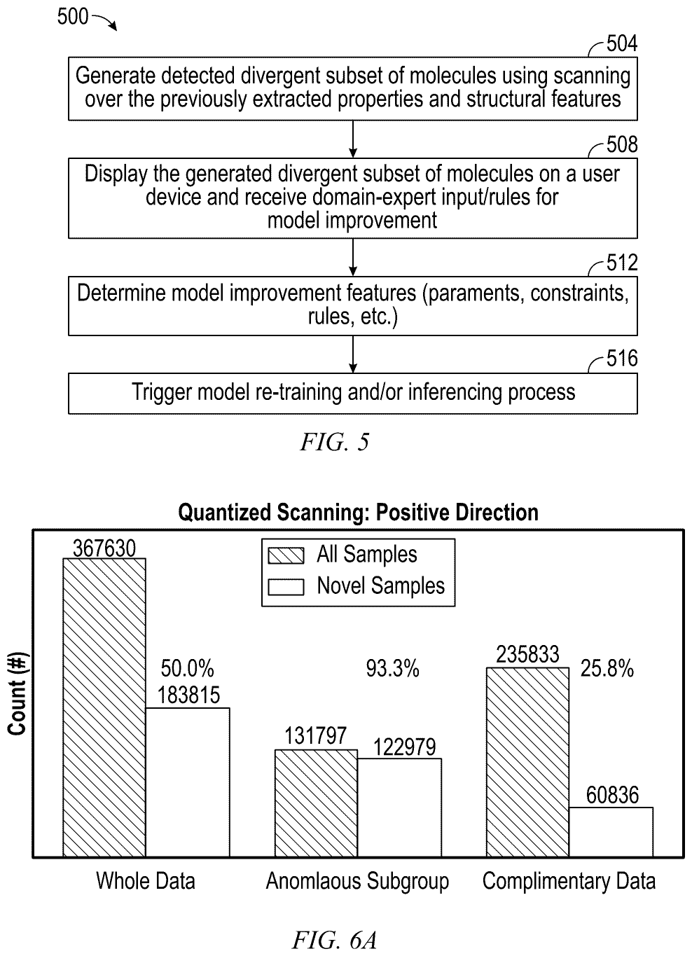

In one set of validation experiments, a trained model was found to generate molecules 224-1, . . . , 224-K with a least likelihood of having an aromatic ring, that is, Aromatic: 0. FIG. 6A is a bar chart illustrating the scanning results on the generated molecules 224-1, . . . , 224-K, in accordance with example embodiments. The generated molecules are determined using a function HasRing (a function of how many rings are counted in the generated molecule; this is a type of molecular property that can be calculated, as will be apparent to the skilled artisan): (−0.001, 4.0] and Aromatic: 0.

FIG. 6B is a table illustrating the results of a comparative evaluation of training a conventional graph convolutional policy network (GCPN) and a conventional flow-based autoregressive model for molecules per their quantum electrodynamic (QED) values, in accordance with example embodiments. As illustrated in FIG. 6A, the training data includes 367,630 samples of which 183,815 are novel. Of the 131,797 anomalous (novel) samples, 122,979 samples (93.3% of the total set of anomalous samples) were discovered using the disclosed techniques to have the desired characteristics. In FIG. 6A, complimentary data refers to samples that do not contain the elements found in the anomalous subgroup, i.e., the whole data excluding the anomalous groups (the first two sets of bar plots in FIG. 6A).

FIG. 6B shows the sets of properties found in molecules generated by different models and training/real molecules that have higher and lower QED, in accordance with an example embodiment. The customized multi-dimensional subset scanning method of FIG. 3B was used to determine the properties of molecules that have a higher QED.

FIG. 7 is a graph providing a visualization of the quantum electrodynamics (QED) distributions of training with real molecules and molecules generated from GCPN and Graph models, in accordance with example embodiments. FIG. 7 illustrates how the domain expert/user compares to generative models based on a single property, in this case, QED. It is observed that a conventional flow-based autoregressive model for molecular graph generation generates a similar distribution to the existing molecules, while GCPN tends to generate molecules with higher QED.

FIG. 8A is a table of Hierarchical Independence Evaluation (HIE) scores for GCPN models and conventional flow-based autoregressive models for molecular graph generation trained on a first conventional dataset, in accordance with example embodiments. Five ranges of the molecular weight in FIG. 8A resulted from the discretization of the feature necessary for sub-feature level independence score (SIS) evaluations. Similar to the tables of FIG. 8A, the comparison is performed between generative models and with the training dataset. Results demonstrate that GCPN and the conventional flow-based autoregressive models for molecular graph generation generated molecules with similar weight characteristics but distinctively different at a few particular ranges, i.e., GCPN generated molecules similar to ZINC-250K with range [258.1, 308.39) whereas the conventional flow-based autoregressive models for molecular graph generation showed better resemblance with ZINC-250K at ranges [201.63, 258.1) and ≥361.47 Daltons. Divergent sub-level generation characteristics are also encoded by lower G vs. G independence score at those ranges.

FIG. 8B is a table of Hierarchical Independence Evaluation (HIE) scores for GCPN models and conventional flow-based autoregressive models for molecular graph generation trained on a second conventional dataset, in accordance with example embodiments. Comparisons between the table of FIG. 8A and the table of FIG. 8B show, using a conventional benchmark platform for training, the conventional flow-based autoregressive models for molecular graph generation achieved more independence than when a conventional dataset is used (compared to GCPN).

Given the discussion thus far, it will be appreciated that, in general terms, an exemplary method, according to an aspect of the invention, includes the operations of extracting, using at least one hardware processor, domain-specific properties from a plurality of datasets related to input molecules 216 and generated molecules 224-1, . . . , 224-K; evaluating, using the at least one hardware processor, a similarity of a given one of the generated molecules 224-1, . . . , 224-K with a candidate molecule; aggregating, using the at least one hardware processor, the evaluated similarity across multiple constraints to generate a single score; quantifying, using the at least one hardware processor, an ability of a given generative model 220-1, 220-2, . . . , 220-K to mimic the input data 216 based on an aggregation of scores for multiple properties; ranking 284, using the at least one hardware processor, the generative models 220-1, 220-2, . . . , 220-K by their ability to mimic the input data 216 and evaluation properties based on the aggregated scores; quantifying, using the at least one hardware processor, one or more rates in the generated molecules 224-1, . . . , 224-K based on the aggregated single score; selecting, using the at least one hardware processor, one of the generative models 220-1, 220-2, . . . , 220-K based on the ranking; and generating, using the at least one hardware processor, a new molecule using the selected generative model 220-1, 220-2, . . . , 220-K. As used herein, a candidate molecule is a molecule of the input molecules 216 being mimicked by the given generative model 220-1, 220-2, . . . , 220-K.

In one example embodiment, the candidate molecule is one of the input molecules 216 or one of the generated molecules 224-1, . . . , 224-K that is generated from a different generative model of the generative models 220-1, 220-2, . . . , 220-K than the given generative model 220-1, 220-2, . . . , 220-K.

In one example embodiment, the evaluating of the similarity is based on a property of the corresponding molecule 216, 224-1, . . . , 224-K, a structural feature of the corresponding molecule 216, 224-1, . . . , 224-K, or both.

In one example embodiment, the quantifying of the ability uses one of a normalized odds ratio and a distribution-similarity metric.

In one example embodiment, the properties are chemical properties, biological properties or both.

In one example embodiment, the multiple constraints are one or more of chemical properties and structural details.

In one example embodiment, each dataset of the plurality of datasets comprises different modalities of the input molecules 216 and the generated molecules 224-1, . . . , 224-K.

In one example embodiment, the extracting of the domain-specific properties is performed using one or more pretrained modality-specific preprocessing machine learning models.

In one example embodiment, the one or more modality-specific preprocessing machine learning models includes a quantization of continuous features.

In one example embodiment, a normalized odds ratio is computed, wherein the normalized odds ratio quantifies the similarity of a generated molecule 224-1, . . . , 224-K with one of the input molecules 216.

In one example embodiment, a normalized distribution-based evaluation technique for non-binary features is customized, wherein the evaluating the similarity is based on the normalized distribution-based evaluation technique.

In one example embodiment, the ranking of the generative models 220-1, 220-2, . . . , 220-K is based on the corresponding aggregated scores and a list of desired molecule features.

In one example embodiment, input from a domain expert is incorporated to improve a generation capability by exploiting generated insights on the generative models 220-1, 220-2, . . . , 220-K and the input molecules 216, the generated molecules 224-1, . . . , 224-K or both.

In one example embodiment, the aggregating the similarity is performed across different features using informed-weighting of feature-based metrics.

In one example embodiment, evaluation results are displayed to an end-user, the evaluation results are transmitted to another system or both (operation 436).

In one example embodiment, a detected divergent subset of molecules is generated using scanning over the extracted domain-specific properties (operation 504); the generated divergent subset of molecules is displayed on a user device (operation 508); domain-expert input is received for model improvement (operation 508); model improvement features are determined based on the domain-expert input for model improvement (operation 512); and a model retraining process, inferencing process or both are triggered (operation 516).

In one example embodiment, a physical molecule is synthesized from a design corresponding to the new molecule.

In one example embodiment, the rates are one or more of validity, uniqueness, and novelty.

In one aspect, a computer program product comprises one or more tangible computer-readable storage media and program instructions stored on at least one of the one or more tangible computer-readable storage media, the program instructions executable by a processor, the program instructions comprising extracting domain-specific properties from a plurality of datasets related to input molecules 216 and generated molecules 224-1, . . . , 224-K; evaluating a similarity of a given one of the generated molecules 224-1, . . . , 224-K with a candidate molecule; aggregating the evaluated similarity across multiple constraints to generate a single score; quantifying an ability of a given generative model 220-1, 220-2, . . . , 220-K to mimic the input data 216 based on an aggregation of scores for multiple properties; ranking 284 the generative models 220-1, 220-2, . . . , 220-K by their ability to mimic the input data 216 and evaluation properties based on the aggregated scores; quantifying one or more rates in the generated molecules 224-1, . . . , 224-K based on the aggregated single score; selecting one of the generative models 220-1, 220-2, . . . , 220-K based on the ranking; and generating a new molecule using the selected generative model 220-1, 220-2, . . . , 220-K.

In one aspect, an apparatus comprises a memory and at least one processor, coupled to the memory, and operative to perform operations comprising extracting domain-specific properties from a plurality of datasets related to input molecules 216 and generated molecules 224-1, . . . , 224-K; evaluating a similarity of a given one of the generated molecules 224-1, . . . , 224-K with a candidate molecule; aggregating the evaluated similarity across multiple constraints to generate a single score; quantifying an ability of a given generative model 220-1, 220-2, . . . , 220-K to mimic the input data 216 based on an aggregation of scores for multiple properties; ranking 284 the generative models 220-1, 220-2, . . . , 220-K by their ability to mimic the input data 216 and evaluation properties based on the aggregated scores; quantifying one or more rates in the generated molecules 224-1, . . . , 224-K based on the aggregated single score; selecting one of the generative models 220-1, 220-2, . . . , 220-K based on the ranking; and generating a new molecule using the selected generative model 220-1, 220-2, . . . , 220-K.

Refer now to FIG. 9.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as a system 200 that performs model-agnostic evaluation of a generative model in a materials discovery processes or the like in accordance with aspects of the invention. In one or more embodiments, system 200 improves a generation process for molecules that aims to characterize and quantify how capable a generative model is for generating a certain type of candidate molecule for a given task and, with this information, informs a user which model to use for a given type of properties needed in the generated molecule as well as informing the models to use during inference time to control the generation process. System 200 can include code for carrying out the AI computational chemistry, and optionally control chemical process equipment to synthesize the molecules. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
extracting, using at least one hardware processor, domain-specific properties from a plurality of datasets related to input molecules and generated molecules;
evaluating, using the at least one hardware processor, a similarity of a given one of the generated molecules with a candidate molecule;
aggregating, using the at least one hardware processor, the evaluated similarity across multiple constraints to generate a single score;
quantifying, using the at least one hardware processor, an ability of a given generative model to mimic the input molecules based on an aggregation of scores for multiple properties;
ranking, using the at least one hardware processor, the generative models by their ability to mimic the input molecules and evaluation properties based on the aggregated scores;
quantifying, using the at least one hardware processor, one or more rates in the generated molecules based on the aggregated single score;
selecting, using the at least one hardware processor, one of the generative models based on the ranking; and
generating, using the at least one hardware processor, a new molecule using the selected generative model.

2. The method of claim 1, wherein the candidate molecule is one of the input molecules or one of the generated molecules that is generated from a different generative model of the generative models than the given generative model.

3. The method of claim 1, wherein the evaluating of the similarity is based on a property of the corresponding molecule, a structural feature of the corresponding molecule, or both.

4. The method of claim 1, wherein the quantifying of the ability uses one of a normalized odds ratio and a distribution-similarity metric.

5. The method of claim 1, wherein the properties are chemical properties, biological properties or both.

6. The method of claim 1, wherein the multiple constraints are one or more of chemical properties and structural details.

7. The method in claim 1, wherein each dataset of the plurality of datasets comprises different modalities of the input molecules and the generated molecules.

8. The method of claim 1, wherein the extracting of the domain-specific properties is performed using one or more pretrained modality-specific preprocessing machine learning models.

9. The method of claim 8, wherein the one or more modality-specific preprocessing machine learning models includes a quantization of continuous features.

10. The method of claim 1, further comprising computing a normalized odds ratio, wherein the normalized odds ratio quantifies the similarity of a generated molecule with one of the input molecules.

11. The method of claim 1, further comprising customizing a normalized distribution-based evaluation technique for non-binary features, wherein the evaluating the similarity is based on the normalized distribution-based evaluation technique.

12. The method of claim 1, wherein the ranking of the generative models is based on the corresponding aggregated scores and a list of desired molecule features.

13. The method of claim 1, further comprising incorporating input from a domain expert to improve a generation capability by exploiting generated insights on the generative models and the input molecules, the generated molecules or both.

14. The method of claim 1, wherein the aggregating the similarity is performed across different features using informed-weighting of feature-based metrics.

15. The method of claim 1, further comprising displaying evaluation results to an end-user, transmitting the evaluation results to another system or both.

16. The method of claim 1, further comprising:
generating a detected divergent subset of molecules using scanning over the extracted domain-specific properties;
displaying the generated divergent subset of molecules on a user device;
receiving domain-expert input for model improvement;
determining model improvement features based on the domain-expert input for model improvement; and
triggering a model retraining process, inferencing process or both.

17. The method of claim 1, further comprising synthesizing a physical molecule from a design corresponding to the new molecule.

18. The method of claim 1, wherein the rates are one or more of validity, uniqueness, and novelty.

19. A computer program product, comprising:
one or more tangible computer-readable storage media and program instructions stored on at least one of the one or more tangible computer-readable storage media, the program instructions executable by a processor, the program instructions comprising:
extracting domain-specific properties from a plurality of datasets related to input molecules and generated molecules;
evaluating a similarity of a given one of the generated molecules with a candidate molecule;
aggregating the evaluated similarity across multiple constraints to generate a single score;
quantifying an ability of a given generative model to mimic the input molecules based on an aggregation of scores for multiple properties;
ranking the generative models by their ability to mimic the input molecules and evaluation properties based on the aggregated scores;
quantifying one or more rates in the generated molecules based on the aggregated single score;
selecting one of the generative models based on the ranking; and
generating a new molecule using the selected generative model.

20. A system comprising:
a memory; and
at least one processor, coupled to said memory, and operative to perform operations comprising:
extracting domain-specific properties from a plurality of datasets related to input molecules and generated molecules;
evaluating a similarity of a given one of the generated molecules with a candidate molecule;
aggregating the evaluated similarity across multiple constraints to generate a single score;
quantifying an ability of a given generative model to mimic the input molecules based on an aggregation of scores for multiple properties;
ranking the generative models by their ability to mimic the input molecules and evaluation properties based on the aggregated scores;
quantifying one or more rates in the generated molecules based on the aggregated single score;
selecting one of the generative models based on the ranking; and
generating a new molecule using the selected generative model.
